(19) United States
(12) Patent Application Publication
(10) Pub. No.: US 2010/0189780 A1
(43) Pub. Date: Jul. 29, 2010

(54) Novel Powderous Medicaments Comprising Tiotropium and Salmeterol and Lactose as Carrier

(75) Inventors: Michael Walz, Bingen (DE); Stephanie Ossadnik, Ingelheim (DE); Michael Trunk, Ingelheim (DE); Christoph Kreher, Ingelheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/670,001

(22) PCT Filed: Jul. 18, 2008
(86) PCT No.: PCT/EP08/59465

§ 371 Date: Apr. 13, 2010

Related U.S. Application Data
(60) Provisional application No. 61/051,933, filed on May 9, 2008.

(30) Foreign Application Priority Data
Jul. 21, 2007 (DE) 102007034157

(51) Int. Cl. A61K 9/48; A61K 31/46; A61K 9/14; A61P 11/00; B65D 81/26; B65D 85/00; B65B 31/00; A61M 15/00

(52) U.S. Cl. 424/452; 514/291; 424/489; 206/204; 206/461; 53/410; 128/203.12

(57) ABSTRACT

The invention relates to stable preparations in powder form for inhalation comprising a tiotropium salt and salmeterol xinafoate, process for the production thereof, and the use thereof for manufacturing a medicament for the treatment of respiratory disorders, especially for the treatment of COPD (chronic obstructive pulmonary disease) and asthma.

The present invention relates to stable medicament compositions for use in inhalation, which contain a combination of tiotropium salts 1 with salmeterol salts 2 as a preparation with lactose. In addition the invention relates to processes for their production as well as their use for the production of a medicament for treating respiratory tract diseases, in particular for treating COPD (chronic obstructive pulmonary disease) and asthma.

### BACKGROUND OF THE INVENTION

Both tiotropium salts 1 and also salmeterol salts 2 are known from the prior art and are both used in the treatment of respiratory tract diseases.

Tiotropium bromide is known from European Patent Application EP 418 716 A1 and has the following chemical structure:

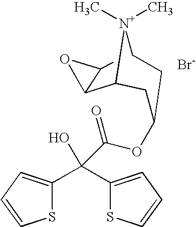

Also, the combination of tiotropium salts 1 and salmeterol salts 2 is known in the prior art. This together with other medicament combinations of long-acting beta mimetics with long-acting anticholinergics is disclosed in WO 00/69468.

Pulverulent preparations of medicaments for inhalation that include the combination of salmeterol xinafoate and tiotropium salts are disclosed in WO2004/058233. The formulations disclosed there are characterised by a high degree of homogeneity and uniformity.

Powder inhalants are packed for example as inhalation powders in suitable capsules and administered by means of powder inhalers. Other systems, in which the amount of powder to be applied is predosed (e.g. blisters) as well as multidose powder systems, are also known. Conventional methods for producing powder inhalants, for example in the form of capsules for inhalation, are described e.g. in DE-A-179 22 07. A further important aspect of powder inhalants is that in the inhalative administration of the active constituent only particles of a certain aerodynamic size reach the target organ, i.e. The lungs. The particle size of these particles accessible to the lungs (inhalable fraction) is in the region of a few μm, typically between 1 and 10 μm, preferably between 1 and 6 μm. Such particles are normally produced by micronising (air jet grinding).

Packaging units for pharmaceutical products that are intended to ensure the maintenance of specific properties of inhalable medicament preparations are described in general terms in the literature. The pharmaceutical active constituents, for example in the form of capsules or tablets, are in this connection often packaged in blister packs, in which the cavities of the blister packs protect the active constituent against external environmental influences. In order to protect the contents against the influence of moisture, such packaging units can additional contain desiccants. Such a packaging unit is disclosed for example in the form of a collapsible cardboard box containing blister packs in EP 0479282 A1.

When using conventional desiccants, such as for example silica gel or molecular sieves (zeolites), an uncontrollable residual moisture is present in the ambient surroundings, for example in a flexible tubular bag of an aluminium composite film or in a HD-PE bottle, which can be a constituent of a packaging unit for pharmaceutical products. This moisture depends on the type of desiccant, the already present water content of the desiccant, the amount of desiccant and the existing sources of water, for example the moisture content of the packaging substances, medicament and trapped air, but also on the water that penetrates during storage.

As a rule a significant excess of desiccant is added to the packaging in order to achieve in any case a reliable drying effect. Such a packaging unit that contains a desiccant for completely removing the ambient moisture is disclosed for example in WO 2004/105727 A2. If unconditioned/untreated desiccants are used, then a residual moisture (humidity) of less than 2% relative humidity can be achieved.

It has surprisingly been found that the procedures known from the prior art are not suitable for preparing inhalation powders that contain, apart from tiotropium salts and salmeterol, the excipient lactose and which are characterised by a sufficient degree of stability.

Within the scope of the present invention stable inhalation powders are understood to be those inhalation powders whose properties remain unchanged even over a relatively long period of time. The properties of inhalation powders do not change if the chemical stability of the individual components in the powder mixture as well as their physical and physicochemical stability is ensured. This also presupposes that the components of the powder mixture remain unchanged as regards their polymorphic and morphological properties.

An object of the present invention is to provide pulverulent medicament preparations for inhalation that contain, apart from a tiotropium salt 1 and a salmeterol salt 2 also lactose as excipient and that are characterised by a high degree of stability.

A further object of the present invention is to provide pulverulent medicament compositions of the aforementioned type whose use allows the inhalation of active constituents with a high inhalable fraction.

The inhalable fraction is the amount of inhalable active constituent particles (<5 μm), as can be determined on the basis of Pharm. Eur. 2.9.18 (European Pharmacopoeia, 6th Edition 2008, Apparatus D—Andersen Cascade Impactor) and USP30-NF25 <601>. The inhalable fraction is also termed within the scope of the present invention as the FPD (fine particle dose).

A further object of the present invention is to prepare pulverulent medicament compositions of the aforementioned type that are characterised by a high fine particle fraction (FPF). In this connection the FPF is the relative FPD referred to the nominal dose per application. The FPF can thus be obtained by determining the FPD according to the above procedure, and this is referred to the nominal dose (given in [%]).

A particular object of the present invention is to provide pulverulent medicament compositions of the aforementioned type that are characterised in that the FPF exhibits a high constancy even on storage over a fairly long period of time, preferably 18 months (storage conditions according to ICH Guideline, 25° C. and 60% relative humidity).

Moreover, a further object of the present invention is to provide inhalation powders whose inhalable fraction is largely independent of the flow rate during discharge (discharge based on the determination of the FPD, but with a variable flow: 20 L/min, 30 L/min, 40 L/min, 60 L/min)

The object of the present invention is also to provide processes for producing medicament preparations according to the invention.

### DETAILED DESCRIPTION OF THE INVENTION

It has surprisingly been found that the objects mentioned in the introduction are achieved by pulverulent preparations according to the invention for inhalation (inhalation powders), which contain apart from a tiotropium salt 1 also a salmeterol salt 2 and the excipient lactose, and which are characterised by an a_{W} value of between 0.05 and 0.5.

The a_{W} value (also termed water activity) is understood in this connection to be a measure of freely available water in a material. This is defined as the quotient of the water vapour pressure (p) above a material, in this case the medicament preparation, and the water vapour pressure above pure water (p0) at a specified temperature, in this case 25° C., in equilibrium:

a_{W}−value=p/p0.

Medicament preparations according to the invention are also characterised by the fact that they have an a_{W} value between 0.05 and 0.5, preferably between 0.10 and 0.45, particularly preferably between 0.10 and 0.40, especially preferably between 0.15 and 0.40 and most particularly preferably between 0.15 and 0.35. According to the invention it is possible in this way for the medicament preparations to be brought by means of a conditioning step into equilibrium with the relative humidity prevailing directly above the product, so that an a_{W} value between 0.05 and 0.5, preferably between 0.10 and 0.45, particularly preferably between 0.10 and 0.40, especially preferably between 0.15 and 0.40 and most particularly preferably between 0.15 and 0.35 is established.

The characteristic a_{W} value for the medicament preparations according to the invention characterises in this connection the medicament preparations that exist after the production of the bulkware, as well as after their packaging and also during their shelf life until the removal of the medicament from the packaging means (in the context of the prescribed use as a medicament).

In the context of the present invention the salmeterol salt 2 is contained in the form of an acid addition salt. Particularly preferred inhalation powders contain as salmeterol salt 2 salmeterol xinafoate (i.e.: (R,S)-4-hydroxy-α1-[[[6-(4-phenylbutoxy)hexyl]amino]methyl]-1,3-benzenedimethanol 1-hydroxy-2-naphthalenecarboxylate). Salmeterol 2′ is understood to mean the free salmeterol base.

In the context of the present invention tiotropium salts 1 are understood to mean salts that are formed from the pharmacologically active cation tiotropium 1′. In the context of the present patent application an explicit reference to the cation tiotropium is indicated by the use of the designation 1′. Tiotropium 1′ is understood to mean the free ammonium cation. If in the context of the present invention the designation 1 is used, this is understood to refer to tiotropium in combination with a corresponding counterion. Suitable counterions (anion) are preferably chloride, bromide, iodide, methanesulfonate or para-toluene sulfonate. Of these anions, bromide is preferred. The hydrates of tiotropium bromide are preferably used for the preparation of the tiotropium-containing inhalation powders according to the invention. In this case the crystalline tiotropium bromide monohydrate known from WO 02/30928 is particularly preferably used. This crystalline tiotropium bromide monohydrate is characterised by an endothermal maximum occurring under thermal analysis by means of differential scanning calorimetry (DSC), at 230°±5° C. at a heating rate of 10 K/min. This monohydrate is further characterised by the fact that in the IR spectrum it exhibits bands inter alia at wavelengths of 3570, 3410, 3105, 1730, 1260, 1035 and 720 cm⁻¹. Finally, this crystalline tiotropium bromide monohydrate forms, as is found by single crystal X-ray structure analysis, a simple monoclinic cell having the following dimensions: a=18.0774 Å, b=11.9711 Å, c=9.9321 Å, β=102.691°, V=2096.96 Å³.

The active constituents mentioned above are used according to the invention in the form of their micronisates. Conventional mills can be employed for carrying out the micronising process. Preferably the micronising is carried out with the exclusion of moisture, particularly preferably using a suitable inert gas, such as for example nitrogen. The use of air jet mills has proved particularly advantageous, in which the comminution of the grinding material takes place by impact of the particles on one another as well as impact of the particles on the walls of the vessel containing the grinding material. According to the invention nitrogen is preferably used as grinding gas. The grinding material is conveyed by means of the grinding gas under specific pressures (grinding pressure). In the context of the present invention the grinding pressure is normally adjusted to a value between about 2 and about 8 bar, preferably between about 3 and about 7 bar, particularly preferably between about 3.5 and about 6.5 bar. The addition of the grinding material to the air jet mill is carried out by means of a feed gas under specific pressures (feed pressure). In the context of the present invention a feed pressure between about 2 and about 8 bar, preferably between about 3 and about 7 bar, particularly preferably between about 3.5 and about 6 bar, has proved suitable. An inert gas, particularly preferably nitrogen, is similarly preferably used as feed gas. The grinding material can in this connection be added at a conveying rate of about 3-65 g/min, preferably 5-35 g/min, particularly preferably about 10-30 g/min.

The medicament preparations according to the invention are furthermore characterised by the fact that they contain lactose as pharmaceutically compatible excipient. In the context of the invention lactose monohydrate is particularly preferably used as excipient.

It is particularly preferred to use excipients that have a mean particle size of 15 to 65 μm, and in particularly preferred inhalation powders the excipient is characterised by a mean particle size of 20 to 47 μm, particularly preferably of 27 to 45 μm. In this case the term mean particle size in the sense used here is understood to mean the 50% value from the volume distribution, measured with a laser diffractometer according to the dry dispersion method. Particularly preferably in addition those excipients are used that have a 10% fine fraction of 1 to 8 μm. In this connection the term 10% fine fraction as used here is understood to mean the 10% value from the volume distribution measured with a laser diffractometer. In other words, in the context of the present invention the 10% fine fraction value denotes the particle size below which 10% of the amount of particles lie (referred to the volume distribution). Furthermore, in particular those inhalation powders are particularly preferred in which the 10% fine fraction of the excipient is about 2 to 7 μm, preferably about 3 to 6 μm.

Also preferred according to the invention are those inhalation powders in which the excipient has a specific surface between 0.2 and 1.5 m²/g, preferably between 0.3 and 1.2 m²/g, particularly preferably between 0.4 and 1.0 m²/g.

Lactose of high crystallinity is preferably used for the powder formulations according to the invention. This crystallinity can be assessed on the basis of the enthalpy (solution enthalpy) released during the dissolution of the excipient. In the case of the excipient lactose monohydrate that is particularly preferably employed according to the invention, lactose is preferably used that is characterised by a solution enthalpy ≧45 J/g, preferably ≧50 J/g, particularly preferably of ≧52 J/g.

The inhalation powders according to the invention are, corresponding to the object forming the basis of the present invention, characterised by a high degree of homogeneity in the sense of the individual dose accuracy. This lies in the region of <8%, preferably <6%, particularly preferably <4% (relative standard deviation referred to individual dose content determinations).

If necessary it may be helpful to use, as an alternative to the excipients mentioned hereinbefore, excipient mixtures that consist of a mixture of coarser excipient with a mean particle size of 17 to 50 μm, preferably 20 to 40 μm, particularly preferably 25 to 35 μm, and a finer excipient with a mean particle size of 1 to 8 μm, preferably 2 to 7 μm, particularly preferably 3 to 6 μm. Here too the mean particle size is understood to denote the 50% value from the volume distribution measured by means of laser diffraction according to the dry dispersion method. If excipient mixtures mentioned hereinbefore are used, the 10% fine fraction of the coarser excipient component is about 2 to 5 μm, preferably about 3 to 4 μm, and that of the finer excipient component is about 0.5 to 1.5 μm. Inhalation powders are preferred in which the proportion of finer excipient in the overall formulation is 2 to 10%, preferably 3 to 7%, particularly preferably 4 to 6%. Where reference is made in the context of the present invention to the term excipient mixture, then this is always understood to mean a mixture that has been obtained by mixing previously clearly defined components. In the same way, only those mixtures that are obtained by mixing a coarser excipient component with a finer excipient component are understood for example to denote an excipient mixture of coarser and finer excipient fractions.

Accordingly, medicament preparations according to the invention are composed in such a way that they contain 3 μg to 1000 μg, preferably 5 μg to 500 μg, particularly preferably 10 μg to 250 μg, furthermore preferably 15 μg to 150 μg, according to the invention preferably 20 μg to 100 μg, and particularly preferably 25 μg to 50 μg of 2 per individual delivery, wherein this active constituent is present homogeneously distributed in a defined amount of lactose, preferably lactose monohydrate, of between 5 mg and 50 mg, for example 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg or any other value between 5 mg and 50 mg. Moreover, the medicament preparations according to the invention contain per individual dose 1 μg to 5000 μg 1, preferably 2 μg to 2000 μg 1, particularly preferably 3 μg to 1000 μg 1, furthermore preferably 4 μg to 500 μg 1, according to the invention preferably 5 μg to 250 μg 1, moreover preferably 6 μg to 100 μg 1 and particularly preferably 7 μg to 25 μg 1.

Medicament preparations according to the invention are preferred that contain per individual delivery the following amounts of 2′ and 1′: 2′ 12.5 μg and 1′ 15 μg in 5 mg lactose, 2′ 25 μg and 1′ 15 μg in 5 mg lactose, 2′ 50 μg and 1′ 15 μg in 5 mg lactose, 2′ 100 μg and 1′ 15 μg in 5 mg lactose, 2′ 12.5 μg and 1′ 10 μg in 5 mg lactose, 2′ 25 μg and 1′ 10 μg in 5 mg lactose, 2′ 50 μg and 1′ 10 μg in 5 mg lactose, 2′ 100 μg and 1′ 10 μg in 5 mg lactose, 2′ 12 μg and 1′ 7.5 μg in 5 mg lactose, 2′ 25 μg and 1′ 7.5 μg in 5 mg lactose, 2′ 50 μg and 1′ 7.5 μg in 5 mg lactose, 2′ 100 μg and 1′ 7.5 μg in 5 mg lactose, 2′ 12.5 μg and 1′ 5 μg in 5 mg lactose, 2′ 25 μg and 1′ 5 μg in 5 mg lactose, 2′ 50 μg and 1′ 5 μg in 5 mg lactose, 2′ 100 μg and 1′ 5 μg in 5 mg lactose, 2′ 12.5 μg and 1′ 3.8 μg in 5 mg lactose, 2′ 25 μg and 1′ 3.8 μg in 5 mg lactose, 2′ 50 μg and 1′ 3.8 μg in 5 mg lactose, 2′ 100 μg and 1′ 3.8 μg in 5 mg lactose, 2′ 12.5 μg and 1′ 15 μg in 10 mg lactose, 2′ 25 μg and 1′ 15 μg in 10 mg lactose, 2′ 50 μg and 1′ 15 μg in 10 mg lactose, 2′ 100 μg and 1′ 15 μg in 10 mg lactose, 2′ 12.5 μg and 1′ 10 μg in 10 mg lactose, 2′ 25 μg and 1′ 10 μg in 10 mg lactose, 2′ 50 μg and 1′ 10 μg in 10 mg lactose, 2′ 100 μg and 1′ 10 μg in 10 mg lactose, 2′ 12.5 μg and 1′ 7.5 μg in 10 mg lactose, 2′ 25 μg and 1′ 7.5 μg in 10 mg lactose, 2′ 50 μg and 1′ 7.5 μg in 10 mg lactose, 2′ 100 μg and 1′ 7.5 μg in 10 mg lactose, 2′ 12.5 μg and 1′ 5 μg in 10 mg lactose, 2′ 25 μg and 1′ 5 μg in 10 mg lactose, 2′ 50 μg and 1′ 5 μg in 10 mg lactose, 2′ 100 μg and 1′ 5 μg in 10 mg lactose, 2′ 12.5 μg and 1′ 3.8 μg in 10 mg lactose, 2′ 25 μg and 1′ 3.8 μg in 10 mg lactose, 2′ 50 μg and 1′ 3.8 μg in 10 mg lactose, 2′ 100 μg and 1′ 3.8 μg in 10 mg lactose, 2′ 12.5 μg and 1′ 15 μg in 15 mg lactose, 2′ 25 μg and 1′ 15 μg in 15 mg lactose, 2′ 50 μg and 1′ 15 μg in 15 mg lactose, 2′ 100 μg and 1′ 15 μg in 15 mg lactose, 2′ 12.5 μg and 1′ 10 μg in 15 mg lactose, 2′ 25 μg and 1′ 10 μg in 15 mg lactose, 2′ 50 μg and 1′ 10 μg in 15 mg lactose, 2′ 100 μg and 1′ 10 μg in 15 mg lactose, 2′ 12.5 μg and 1′ 7.5 μg in 15 mg lactose, 2′ 25 μg and 1′ 7.5 μg in 15 mg lactose, 2′ 50 μg and 1′ 7.5 μg in 15 mg lactose, 2′ 100 μg and 1′ 7.5 μg in 15 mg lactose, 2′ 12.5 μg and 1′ 5 μg in 15 mg lactose, 2′ 25 μg and 1′ 5 μg in 15 mg lactose, 2′ 50 μg and 1′ 5 μg in 15 mg lactose, 2′ 100 μg and 1′ 5 μg in 15 mg lactose, 2′ 12.5 μg and 1′ 3.8 μg in 15 mg lactose, 2′ 25 μg and 1′ 3.8 μg in 15 mg lactose, 2′ 50 μg and 1′ 3.8 μg in 15 mg lactose, 2′ 100 μg and 1′ 3.8 μg in 15 mg lactose, 2′ 12.5 μg and 1′ 15 μg in 20 mg lactose, 2′ 25 μg and 1′ 15 μg in 20 mg lactose, 2′ 50 μg and 1′ 15 μg in 20 mg lactose, 2′ 100 μg and 1′ 15 μg in 20 mg lactose, 2′ 12.5 μg and 1′ 10 μg in 20 mg lactose, 2′ 25 μg and 1′ 10 μg in 20 mg lactose, 2′ 50 μg and 1′ 10 μg in 20 mg lactose, 2′ 100 μg and 1′ 10 μg in 20 mg lactose, 2′ 12.5 μg and 1′ 7.5 μg in 20 mg lactose, 2′ 25 μg and 1′ 7.5 μg in 20 mg lactose, 2′ 50 μg and 1′ 7.5 μg in 20 mg lactose, 2′ 100 μg and 1′ 7.5 μg in 20 mg lactose, 2′ 12.5 μg and 1′ 5 μg in 20 mg lactose, 2′ 25 μg and 1′ 5 μg in 20 mg lactose, 2′ 50 μg and 1′ 5 μg in 20 mg lactose, 2′ 100 μg and 1′ 5 μg in 20 mg lactose, 2′ 12.5 μg and 1′ 3.8 μg in 20 mg lactose, 2′ 25 μg and 1′ 3.8 μg in 20 mg lactose, 2′ 50 μg and 1′ 3.8 μg in 20 mg lactose, 2′ 100 μg and 1′ 3.8 μg in 20 mg lactose, 2′ 12.5 μg and 1′ 15 μg in 25 mg lactose, 2′ 25 μg and 1′ 15 μg in 25 mg lactose, 2′ 50 μg and 1′ 15 μg in 25 mg lactose, 2′ 100 μg and 1′ 15 μg in 25 mg lactose, 2′ 12.5 μg and 1′ 10 μg in 25 mg lactose, 2′ 25 μg and 1′ 10 μg in 25 mg lactose, 2′ 50 μg and 1′ 10 μg in 25 mg lactose, 2′ 100 μg and 1′ 10 μg in 25 mg lactose, 2′ 12.5 μg and 1′ 7.5 μg in 25 mg lactose, 2′ 25 μg and 1′ 7.5 μg in 25 mg lactose, 2′ 50 μg and 1′ 7.5 μg in 25 mg lactose, 2′ 100 μg and 1′ 7.5 μg in 25 mg lactose, 2′ 12.5 μg and 1′ 5 μg in 25 mg lactose, 2′ 25 μg and 1′ 5 μg in 25 mg lactose, 2′ 50 μg and 1′ 5 μg in 25 mg lactose, 2′ 100 μg and 1′ 5 μg in 25 mg lactose, 2′ 12.5 μg and 1′ 3.8 μg in 25 mg lactose, 2′ 25 μg and 1′ 3.8 μg in 25 mg lactose, 2′ 50 μg and 1′ 3.8 μg in 25 mg lactose, 2′ 100 μg and 1′ 3.8 μg in 25 mg lactose.

The conditioning step according to the invention for adjusting a specific a_{W} value requires a further humidification or a drying of the medicament preparation depending on the absolute moisture content of the medicament preparation, in which connection in the context of the invention the packed medicament preparation is understood to mean for example the medicament preparation packed in inhalation capsules or in blister cavities.

According to the invention the objectives involved in the process for preparing the medicament preparations according to the invention are achieved if, as a particular implementation of the invention, the moisture content in a packaging unit is purposefully adjusted, wherein before adding for example a desiccant to the packaging unit the desiccant is within the scope of an additional conditioning step exposed to a defined humidity atmosphere with a specific residual humidity.

By means of these measures the humidity within the packaging unit can be controlled within a defined bandwidth over the storage life over a medicament, i.e. the humidity can be reliably prevented from exceeding an upper limiting value as well as falling below a lower limiting value. In this way the medicament can be protected against the negative effects of too high or too low a humidity. Stability requirements, in particular in the case of complex active constituent combinations, can thus be better satisfied. Possible structure changes in some active constituents, which can lead to an altered and thus undesired pharmaceutical action, are avoided.

To prepare the medicaments according to the invention it is first of all necessary to provide the starting materials of the medicament preparations according to the invention as homogeneous powder mixtures. After weighing out the starting materials the preparation of the powder mixtures from the excipient and the active constituent or active constituents is carried out using methods known in the prior art. In this connection reference may be made for example to the disclosures in WO 02/30390 and WO2004/058233. The inhalation powders according to the invention can accordingly be obtained for example according to the procedure described hereinafter. In the production processes described hereinafter the mentioned components are used in the weight proportions as specified in the previously described compositions of the inhalation powders. Alternatively, the homogeneous powder mixtures can also be produced by means of high-shear mixers (intensive mixers, Diosna mixers) as well as low-shear mixers (screw-type mixers, Ruber mixers). In this connection, ca. ⅓ of the total amount of the excipient (carrier material) lactose can be added beforehand to the mixing vessel. The addition preferably takes place through a sieve or a sieve granulator with a mesh width of 0.1 to 2 mm, particularly preferably 0.3 to 1 mm, most particularly preferably 0.3 to 0.6 mm. As a variant, the sieve/screen can be intermediately flushed with the excipient, provided that the active constituent or active constituents are added in portions. After the addition of the active constituent the sieve is post-flushed with the excipient. An alternating, layer-wise screening of the various components is preferred.

If the active constituents used in the processes described above have not already been obtained after their chemical preparation in a crystalline form that has the aforementioned particle sizes, then they can be converted by grinding into the particle sizes that satisfy the parameters mentioned hereinbefore (so-called micronising). Appropriate micronising methods are known from the prior art (for example WO2004/058233). One possible embodiment of an air jet mill that has proved particularly suitable for grinding the salmeterol salts 2 and without restricting the subject-matter of the invention is for example the following equipment: Chrispro Jet-Mill MC200, grinding chamber size=200 mm with 2.25 mm grinding dies, Firma Micro Macinazione SA, Via Cantonale, 6995 Molinazzo di Monteggio (CH). In this connection it has proved particularly suitable for the micronising of the salmeterol salts 2 if the grinding pressure is adjusted to a value between about 2 and about 9 bar, preferably between about 3 and about 6 bar, particularly preferably between about 3.5 and about 4.5 bar. The addition of the grinding material to the air jet mill is carried out by means of the feed gas under specific pressures (feed pressure). In the context of the present invention a feed pressure between about 2.5 and about 9.5 bar, preferably between about 3.5 and about 6.5 bar, particularly preferably between about 4 and about 5 bar, has proved suitable. Likewise an inert gas, particularly preferably nitrogen, is preferably used as feed gas. The addition of the grinding material (preferably crystalline salmeterol xinafoate) can in this connection take place at a conveying rate of about 100-300 g/min, preferably about 150-250 g/min.

To carry out the process, alternatively conventional air jet mills as well as counter-jet mills can be used. Preferably the micronising is carried out under the exclusion of moisture, particularly preferably using a suitable inert gas, such as for example nitrogen. It has proved particularly preferable to use air jet mills in which the comminution of the grinding material is effected by impact of the particles on one another as well as the impact of the particles on the walls of the grinding vessel. According to the invention nitrogen is preferably used as grinding gas. The grinding material is comminuted by means of the grinding gas under specific pressures (grinding pressure).

Optionally the grinding material thereby obtained can then be processed further under the specific conditions mentioned hereinafter. For this purpose the micronisate is exposed at a temperature of 15°-40° C., preferably 20°-35° C., particularly preferably 25°-30° C., to water vapour with a relative humidity (RH) of at least 40%. Preferably the humidity is adjusted to a value of 50-95 RH, preferably to 60-90% RH, particularly preferably to 70-80% RH. The term relative humidity (RH) is understood here to mean the quotient of the partial pressure of water vapour and the partial pressure of water at the relevant temperature. Preferably the micronisate obtained from the grinding process described hereinbefore is exposed to the spatial conditions mentioned above for a period of 6 hours. However, the micronisate is preferably exposed to the aforementioned spatial conditions for about 12 to about 48 hours, preferably about 18 to about 36 hours, particularly preferably about 20 to about 28 hours.

The micronisate of the tiotropium salt 1, preferably of tiotropium bromide, obtained in a similar way to the aforedescribed procedure—reference may be made here to WO2004/058233—has a mean particle size of between 0.5 μm and 10 μm, preferably between 1 μm and 6 μm, particularly preferably between 2 μm and 5 μm, and a Q_{(5.8)} of greater than 60%, preferably greater than 70%, and particularly preferably greater than 80%. Here the characteristic value Q_{(5.8)} denotes the number of particles lying below 5.8 μm, referred to the volume distribution of the particles. The particle sizes were determined in the context of the present invention by means of laser diffraction (Fraunhofer diffraction). Relevant details can be found in the experimental description of the invention.

A further characteristic property of the tiotropium micronisate preferably used according to the invention and that has been prepared according to the above process are specific surface values in the range between 2 m²/g and 5 m²/g, especially values between 2.5 m²/g and 4.5 m²/g, and particularly especially values between 3.0 m²/g and 4.0 m²/g.

Salmeterol salt micronisate 2 has a mean particle size of between 0.5 μm and 10 μm, preferably between 1 μm and 6 μm, particularly preferably between 1.5 μm and 4.0 μm and a Q_{(3,0)} value between 50% and 90%, preferably between 60% and 80%, particularly preferably between 65% and 75%. In this connection the characteristic value Q_{(3,0)} denotes the number of particles which, referred to the volume distribution of the particles, lie below 3.0 μm. The particle sizes were determined in the context of the present invention by means of laser diffraction (Fraunhofer diffraction). Relevant details can be found in the experimental description of the invention.

Also characteristic of the salmeterol salt micronisate preferably used according to the invention and that has been prepared by the above process are specific surface values in the range between 5 m²/g and 10 m²/g, particularly values between 5.5 m²/g and 9 m²/g and especially values between 6.0 m²/g and 8 m²/g.

As a preferred variant the production process for preparing a micronisate before the latter is processed further, i.e. before the preparation of the powder mixture, can include a further production step. In this case the micronised active constituents to be used having the structure 1 and/or 2, are passed through an ultrasound sieve with a mesh width of 80 μm or 63 μm or 40 μm or 25 μm, or through a sieve with a mesh width of between 25 μm and 80 μm.

The powder formulations according to the invention are preferably used as predosed medicament preparations. For this purpose powder mixtures according to the invention can be predosed according to methods known in the prior art. It is preferred in this connection to use a defined amount for the filling, which can be between 5 mg and 50 mg, for example 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg or any other value between 5 mg and 50 mg. According to the invention a dose receptacle in the form of a cavity for accommodating an amount of powder that is administered as a single dose is preferred.

It has surprisingly been shown that, following the filling step, an equilibration step/conditioning step is found to be advantageous. This is understood according to the invention to denote a process step in which the water content of the product is altered in such a way that as a consequence of this process step it has an a_{W} value between 0.05 and 0.5, preferably between 0.10 and 0.45, particularly preferably between 0.10 and 0.40, especially preferably between 0.15 and 0.40 and most especially preferably between 0.15 and 0.35.

Technically this means that either further water is added to or removed from the product by means of the conditioning step, depending on the absolute water content of the product. In this connection it has been found that by means of a one-step process the target moisture according to the invention can be achieved independently of the actual state of the product after filling the receptacle with the inhalation powder, if the product is exposed to a specific relative humidity RH at a temperature T over a time t. Suitable process parameters are, if the process is carried out at RH=15-35%, T=15-30° C. and t>4 hours, preferably at RH=18-27%, T=16-28° C. and t>4 hours, particularly preferably at RH=19-26%, T=17-27° C. and t>4 hours, most particularly preferably at RH=20-25%, T=19-23° C. and t>4 hours and the medicament preparations according to the invention, for example packed in capsules, are exposed in a closed vessel to these process conditions. In this connection according to the invention it should be ensured that the process parameters RH and T are maintained and monitored within the specified range during the implementation of the process. The relative humidity (RH) is understood here to denote the quotient of the partial pressure of the water vapour and of the vapour pressure of water at the relevant temperature. It is found in this connection that prolonging the process time t while simultaneously maintaining the possible process parameters RH and T according to the above figures at t>8 hours, t>12 hours, t>24 hours and t>48 hours, is preferred for the implementation of the process.

Possible processes, which however should not be regarded as an exhaustive list for the implementation of the above process step, can be designed in such a way that packed medicament preparations according to the invention in a container are exposed to indirect contact, i.e. without touching the product, an exchange of moisture occurring via the gaseous phase, to a preconditioned desiccant. In this case an unlimited gas exchange occurs between the medicament preparation and the preconditioned desiccant. In a preparatory step the desiccant should be brought into equilibrium according to the values of the above process parameters RH, T and t, by means of commercially available climatic chambers (e.g. Weiss climatic chambers, Weiss Klimatechnik GmbH, 35447 Reiskirchen-Lindenstruth, Germany). Commercially available desiccants such as for example silica gel, molecular sieves (zeolites), and bentonite can be used for this purpose, silica gel being preferred. Alternatively it is also possible to pass a gas stream that corresponds to the above process parameters for RH, T and t through the medicament preparations according to the invention packaged in a container. Furthermore, an alternative method is possible in which the product is exposed directly, for example spread out on trays, to the climatic conditions according to the above process parameters in an adequately controllable climatic chamber while maintaining the above process parameters for RH, T and t.

According to the invention it is preferred to carry out a predosing of the medicament preparation in a dose container that is made of a material that comprises, at least on the contact surface between the inhalation powder, a material that is chosen from the group of synthetic plastics. Also, according to the invention dose containers are preferred that are chosen from a material which can be characterised by the fact that it is not hygroscopic. This is understood to mean the ability to absorb and bind moisture from the ambient atmosphere (generally in the form of water vapour from the atmospheric humidity) and to be able to release the moisture extremely quickly if required. In the context of the present invention a material is regarded as non-hygroscopic if it can absorb and release less than 0.5% (w/w), preferably less than 0.2% (w/w), most preferably less than 0.1% (w/w) of water at a temperature of 25° C., referred to an equilibrium ambient humidity of 5% relative humidity compared to 75% relative humidity. Appropriate measurement methods are known to the person skilled in the art, and a typical example of a possible measurement system is the DVS system from Porotec GmbH (D-65719 Hofheim, Germany). According to the invention, also preferred are dose containers that are made of a material which is characterised in that it has an electrical conductivity sigma that is less than 10⁻⁵ S cm⁻¹, preferably less than 10⁻¹⁰ S cm⁻¹.

As a preferred predosed medicament preparation there should be mentioned filled capsules that contain the inhalation powders according to the invention. The empty capsules are filled with the inhalation powders according to the invention by methods known in the prior art. For this purpose there are particularly preferably used capsules whose material is chosen from the group of synthetic plastics, particularly preferably chosen from the group consisting of polyethylene, polycarbonate, polyester, polypropylene and polyethylene terephthalate. Polyethylene, polycarbonate or polyethylene terephthalate are particularly preferred as synthetic plastics materials. If polyethylene is used as a particularly preferred capsule material according to the invention, preferably polyethylene having a density between 900 and 1000 kg/m³, preferably of 940-980 kg/m³, particularly preferably of about 960-970 kg/m³ (high-density polyethylene) is used. According to the invention powder reservoirs filled in a product-contacting manner with the medicament preparations according to the invention are equated to the capsules. This is understood to mean that powder reservoirs according to the invention are configured in such a way that at least the material contacting the medicament preparation is chosen from a material of the group comprising synthetic plastics. The synthetic plastics in the context of the invention can be processed in a versatile manner by means of the production process known in the prior art. In the context of the invention injection moulding processing of the plastics is preferred. The injection moulding technique avoiding the use of mould release agents is particularly preferred. This production process is well defined and is characterised by a particularly good reproducibility.

A further aspect of the present invention relates to capsules mentioned hereinbefore that contain inhalation powder according to the invention mentioned hereinbefore. These capsules can contain about 1 to 20 mg, preferably about 3 to 15 mg, particularly preferably about 4 to 12 mg of inhalation powder. Preferred formulations according to the invention contain 4 to 6 mg of inhalation powder. Equally important according to the invention are inhalation capsules that contain the formulations according to the invention in an amount of 8 to 12 mg, particularly preferably 9 to 11 mg.

Predosed medicaments according to the invention should be packaged under controlled process parameters RH and T to ensure that the product corresponds to an a_{W} value according to the invention, wherein according to the invention this is understood as a packaging in a primary packaging means. For example, in this case a packaging in the form of blister packs can be used. Here, the blister serves as primary packaging means. Suitable process parameters are when the process is carried out at RH=15-35%, T=15-30° C., preferably at RH=18-27%, T=16-28° C., particularly preferably at RH=19-26%, T=17-27° C., most particularly preferably at RH=20-25%, T=19-23° C. It has proved advantageous in this connection if the sheets for the production of the blisters, starting from the last production stage of the sheets, as well as during storage, transportation, possible intermediate storage up to their use within the scope of the blister packaging of the medicament according to the invention, are exposed to controlled climatic conditions. Preferred climatic conditions are in this case a temperature in the range from 5° C. to 45° C. and a relative humidity of 10% RH to 60% RH, particularly preferably 5° C. to 40° C. and 10% RH to 50% RH, also particularly preferably 5° C. to 40° C. and 15% RH to 40% RH, more particularly preferably 10° C. to 30° C. and 15% RH to 40% RH, even more particularly preferably 10° C. to 30° C. and 20% RH to 30% RH and most particularly preferably 15° C. to 30° C. and 20% RH to 30% RH

A packaging unit configured as a blister packaging that can be used in the context of the present invention for packaging inhalation capsules that contain the medicament preparations according to the invention, consists as a rule of a cover film and a base film, a plurality of cavities being formed in the said base film. The cover film and the base film can be composed of one or more layers of different or identical materials. The cover film is hermetically connected to the base film for example by bonding, welding or sealing. The cover film and/or the carrier film is as a rule formed as a metal foil and/or plastics film and/or paper film. These materials can be present in several layers. Typical metal foils include for example aluminium foils and aluminium composite foils fabricated from aluminium and for example a plastics material. As material for the plastics films there can be used polyvinyl chloride (PVC), cycloolefin copolymer (COC), polychlorotrifluoroethylene (PCFE), polyethylene (PE), polypropylene (PP), polyethylene terephthalate (PET), polycarbonate (PC), polyester (UP), polyacrylate, polyamide (PA) or other plastics. It is preferred in this connection to use the plastics PE and PP, particularly preferably PP. Often a blister consists of a cover foil of aluminium, which seals the base film for accommodating the pharmaceutical product or active constituent. This thermoformed base film can likewise include an aluminium foil in order to prevent the penetration of water into the cavity for receiving the pharmaceutical product. In order to form a further diffusion barrier and to increase the mechanical stability of the blister, optionally at least the aluminium foil of the base film can be covered on one or both sides with further plastics and/or paper films.

In particular blister packagings having the following layer sequence are provided for use in the context of the present invention. The cover film (foil) is made of aluminium and has a thickness of 10 to 80 micrometers, preferably 20 to 50 micrometers, in particular 30 to 40 micrometers. The cover film is hermetically joined by means of a heat sealing lacquer to the base film containing the cavities. The base film consists, on the side in contact with the product, of a PVC, PP, PE layer or the like in a thickness of between 10 and 200 micrometers, preferably between 15 and 50 micrometers, in particular between 20 and 40 micrometers. This film is joined to an aluminium foil whose thickness is preferably 30 to 60 micrometers, advantageously 35 to 50 micrometers. A polyamide film in a thickness of between 10 and 40 micrometers, preferably 15 to 30 micrometers, adjoins the aluminium foil. In an alternative base film the PVC film on the side facing the product is replaced by a polypropylene film or the like. In a preferred blister packaging the cover film consists of a 38 μm-thick aluminium foil and the heat sealing lacquer. The base film is fabricated on the side facing the pharmaceutical product from a 30 μm-thick PVC film, a 45 μm-thick aluminium foil adjoining the latter, as well as a 20 μm-thick polyamide film on the outside.

It is advantageous if the medicament preparations according to the invention, which are packaged for example in blister packs, are additionally protected by means of an additional pouch for the purpose of long-term storage. This additional protection and therefore consequently the pouch corresponds to a secondary packaging means.

In this connection the pouch can for example be configured as a tubular bag or a four-edged sealed bag in such a way that the surface film and the underneath film are hermetically joined to one another for example by bonding, welding or sealing. The films are as a rule formed as a metal or metal-plastics composite or metal-plastics-paper composite film. These materials can be present in several layers. Typical metal films (foils) include for example aluminium foils and aluminium composite foils, which are made of aluminium and for example a plastics material. As material for the plastics films there can be used polyvinyl chloride (PVC), cycloolefin copolymer (COC), polychlorotrifluoroethylene (PCFE), polyethylene (PE), polypropylene (PP), polyethylene terephthalate (PET), polycarbonate (PC), polyester (UP), polyacrylate, polyamide (PA) or other plastics. Also preferred according to the invention are those films that have a permeation rate for water of less than 5 g/m²d, preferably less than 2 g/m²d, particularly preferably less than 1 g/m²d, most particularly preferably less than 0.1 g/m²d and most especially preferably less than 0.05 g/m²d. In this connection the term permeation rate is understood to be a measure of the water vapour permeability through the film according to DIN 53122 at 38° C. and 90% relative atmospheric humidity.

In this connection it is preferred if the pouch is configured in such a way that a small, pre-moisturised desiccant packet in addition to one or more than one blister card is placed in the said pouch. Such a pre-moisturised desiccant packet can be obtained by an additional conditioning step.

The time needed for this additional conditioning step is, in order to obtain a pre-moisturised desiccant packet, dependent on achieving a humidity equilibrium between the desiccant and the ambient atmosphere. In this connection the residual moisture of the atmosphere is adjusted corresponding to the desired minimal residual moisture in the packaging unit after the packaging of the pharmaceutical active constituent formulation. In this way the desiccant can be preconditioned in a targeted manner corresponding to the optimal storage conditions of the active constituent or active constituent formulation, and the corresponding a_{W} value can be maintained.

In a further modification of the conditioning step for the pre-moisturising of the desiccant packet, a homogeneous distribution of the humidity atmosphere during the additional conditioning step is established above the desiccant. In this way it is ensured that all the desiccant has the same residual moisture content, and that after the packaging no change as regards the desired, pre-adjusted moisture range can occur as a result of compensation/equilibration processes.

This can be achieved effectively and efficiently in terms of the shortest possible process time and homogenisation if the desiccant, for example in the form of loose granules or packed in breathing-active bags, is during the additional conditioning step circulated within the moisture atmosphere, which can take place for example in drum or stirred devices.

A packaging unit for accommodating a pharmaceutical active constituent formulation that additionally contains a desiccant which has been preconditioned by means of the process described hereinbefore, provides the possibility of safely storing medicaments that are particularly sensitive as regards the moisture level.

A pre-moisturised desiccant packet can be understood in this connection to denote for example a packaged amount of silica gel, molecular sieve (zeolite) or bentonite that has a water load. If the desiccant packet according to the invention is placed in a closed container (for example pouch), then this water load means that the relative humidity in this closed container at 25° C. has a value between 10% and 40%. This can be achieved if the aforesaid desiccant packet before its use as an auxiliary component of the packaged medicament preparation according to the invention is preconditioned for example in a climatic chamber at 15-30° C. and 15-35% relative humidity, preferably at 20-28° C. and 15-30% relative humidity, particularly preferably at 23-27° C. and 20-30% relative humidity. Depending on the flow conditions (air exchange rate) of the climatic chamber that is used, as well as the substance-specific sorption kinetics of the desiccant that is used, an equilibrium of the water load of the aforesaid desiccant is normally achieved after 8 hours, preferably after 16 hours, particularly preferably after 24 hours.

Similarly medicament preparations according to the invention can be packaged so that, according to the above process step for the conditioning of the bulkware (e.g. filled inhalation capsules), these are packaged and protected in a HD-PE bottle containing a pre-moisturised desiccant cartridge (process parameters for the pre-moisturising in accordance with the process parameters for the preparation of the desiccant packets that can be placed in pouch packagings according to the invention). Thus, it can also be ensured that the property of the medicament preparation according to the invention is guaranteed with such a packaging configuration.

In a further alternative it is also possible to package in a pouch medicament preparations according to the invention preconditioned and packaged in blisters so as to ensure a suitable a_{W} value (measured directly above the packaged medicament preparation), in which connection the long-term storability is ensured by for example gassing the pouch with conditioned air. For the purposes of gassing such a pouch, according to the invention conditioned air that has for example a water load of 10%-35% relative humidity, referred to 25° C., preferably a water load of 10%-30% relative humidity referred to 25° C., particularly preferably a water load of 10%-25% relative humidity referred to 25° C. and most particularly preferably a water load of 15%-25% relative humidity referred to 25° C., is found to be advantageous. In this connection technically absolute water loads in the conditioned air for the gassing of the pouch, such as are obtained by conversion calculation according to the Mollier diagram from the prior art, can likewise be used according to the invention.

Packaging configurations that ensure that the medicament preparations according to the invention have an a_{W} value of between 0.05 and 0.5, preferably between 0.10 and 0.45, particularly preferably between 0.15 and 0.40 and most particularly preferably between 0.15 and 0.35 during the useful life of the medicament up to and including the removal of the medicament from the packaging means, preferably for a period of at least 18 months storage, are likewise specifically included. Medicament preparations according to the invention thus fulfil the stability requirements at 25° C./18 months of the ICH Guideline Q1A (R2), February 2003.

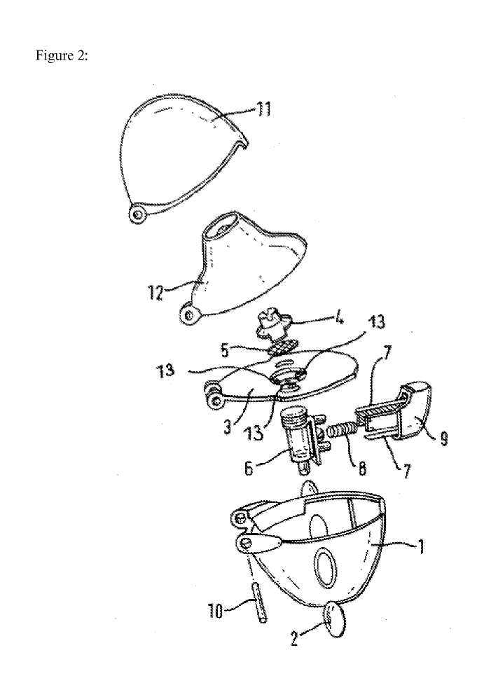

The present invention moreover relates to an inhalation kit consisting of one or more of the aforedescribed capsules characterised by a content of inhalation powder according to the invention, in conjunction with the inhaler according to FIG. 2.

The present invention in addition relates to the use of the capsules mentioned hereinbefore and characterised by a content of inhalation powder according to the invention, for the production of a medicament for treating respiratory tract diseases, in particular for treating COPD and/or asthma.

The present invention additionally relates to the use of the inhalation powders according to the invention for the production of a medicament for treating respiratory tract diseases, in particular for treating COPD and/or asthma, characterised in that the inhaler illustrated in FIG. 2 is used.

### Starting Materials

### I) Excipient:

### Ia.:

In the following examples 1 to 5 lactose monohydrate is used as excipient. This can be obtained for example from the company Borculo Domo Ingredients, Borculo/NL under the product name Lactochem Extra Fine Powder. The specifications according to the invention for the particle size and the specific surface are satisfied by this lactose quality. In addition this lactose has the preferred solution enthalpy values according to the invention and mentioned hereinbefore. For example, lactose batches having the following specifications were used in the following examples:
a) mean particle size: 17.9 μm; 10% fines fraction: 2.3 μm; specific surface: 0.61 m²/g; or
b) mean particle size: 18.5 μm; 10% fines fraction: 2.2 μm; specific surface: 0.83 m²/g;
c) mean particle size: 21.6 μm; 10% fines fraction: 2.5 μm; specific surface: 0.59 m²/g;
d) mean particle size: 16.0 μm; 10% fines fraction: 2.0 μm; specific surface: 0.79 m²/g

### Ib.:

In the following examples 6 to 9 lactose monohydrate (200M) is used as coarser excipient. This can be obtained for example from the company DMV International, 5460 Veghel/NL under the product name Pharmatose 200M. This lactose is characterised by a mean particle size of about 30 to 35 μm. Employed lactose 200M batches have for example a mean particle size of 31 μm with a 10% fines fraction of 3.2 μm or also a mean particle size of 34 μm with a 10% fines fraction of 3.5 μm.

In the following examples 6 to 9 lactose monohydrate with a mean particle size of 3-4 μm is used as finer excipient. This can be obtained by conventional methods (micronising) from commercially obtainable lactose monohydrate, for example the aforementioned lactose 200M. Employed micronised lactose batches have for example a mean particle size of 3.7 μm with a 10% fines fraction of 1.1 μm or also a mean particle size of 3.2 μm with a 10% fines fraction of 1.0 μm.

### Ic.:

In the following examples 10 to 12 lactose monohydrate (200M) is used as excipient. This can be obtained for example from the company DMV International, 5460 Veghel/NL under the product name Pharmatose 200M. This lactose is characterised by a mean particle size of about 30 to 35 μm.

### Id.:

In the following examples 13 to 19 lactose monohydrate (200M) is used as excipient. This can be obtained for example from the company DMV International, 5460 Veghel/NL under the product name Respitose ML003. This lactose is characterised by a mean particle size of about 30 to 35 μm. Employed Respitose ML003 lactose batches have for example a mean particle size of 31 μm with a 10% fines fraction of 3.2 μm or also a mean particle size of 34 μm with a 10% fines fraction of 3.5 μm.

### II) Preparation of Salmeterol Xinafoate According to the Invention:

20 g of salmeterol base and 9.1 g of 1-hydroxy-2-naphthoic acid are suspended in 260 ml of abs. ethanol and 260 ml of tert.-butyl methyl ether. The suspension is heated to 55-56° C. and stirred until a clear solution has formed. The solution is filtered and the filter is rinsed with 30 ml of abs. ethanol and 30 ml of tert.-butyl methyl ether. The filtrate is cooled to 38° C. and seeded with a few crystals of salmeterol zinafoate. The solution is stirred for 1 hour at 34-37° C., whereupon crystallisation commences. The suspension is cooled to 1-3° C. and stirred for ca. 30 minutes at this temperature. The precipitate is filtered through a suction filter and washed with 20 ml of ethanol and 120 ml of tert.-butyl methyl ether. The solid is dried at 45° C. in a stream of nitrogen. Yield: 26 g (89.5%).

The crystalline salmeterol xinafoate thereby obtained has a tamped volume of 0.27 g/cm³.

### III) Micronising of Salmeterol Xinafoate:

### IIIa)

The salmeterol xinafoate obtainable according to the above procedure is micronised with an air jet mill of the MC JETMILL 50 type from the company Jetpharma; Via Sotto Bisio 42 a/c, 6828-Balerna, Switzerland. Using nitrogen as grinding gas, the following grinding parameters are set for example:

Grinding pressure 7.5 bar, feed pressure 8.0 bar.

Addition (of the crystalline salmeterol xinafoate) and flow rate 40 g/min.

### IIIb)

The salmeterol xinafoate obtainable according to the above procedure is micronised with an air jet mill of the Chrispro Jet-Mill MC200 type, grinding chamber size=200 mm, with 2.25 mm grinding nozzles, Firma Micro Macinazione SA, Via Cantonale, 6995 Molinazzo di Monteggio (CH). The grinding process is carried out using this equipment with the following grinding parameters:

Grinding pressure: 4 bar; feed pressure: 4.5 bar: addition of the grinding material: about 200 g/min

The micronised salmeterol xinafoate thereby obtained has a tamped volume of 0.19 g/cm³.

### IV) Micronising of Crystalline Tiotropium Bromide Monohydrate:

The crystalline tiotropium bromide monohydrate obtainable according to WO 02/30928 is micronised with an air jet mill of the 2″ microniser type with a grinding ring of 0.8 mm bore, from the company Sturtevant Inc., 348 Circuit Street, Hanover, Mass. 02239, USA. The following grinding parameters for example are set using nitrogen as grinding gas:

Grinding pressure: 5.5 bar; feed pressure: 5.5 bar;

Addition (of the crystalline monohydrate) and flow rate: 19 g/min.

The obtained ground material is then spread on trays (for example metal tray sheets from Firma Glatt, 79589 Binzen, Germany) in a layer thickness of about 1 cm and exposed for 24-24.5 hours to the following climatic conditions:

Temperature: 25-30° C.; relative humidity: 70-80%.

### Measurement Methods:

### I) X-Ray Structure Analysis of Salmeterol Xinafoate:

### Measuring Instrument and Settings:

The X-ray powder diagram was taken in the context of the present invention using a BRUKER D8 ADVANCED diffractometer equipped with a position-sensitive detector (=PSD) and a Cu anode as X-ray source (CuK_{α} radiation, λ=1.5418 Å, 40 kV, 40 mA).

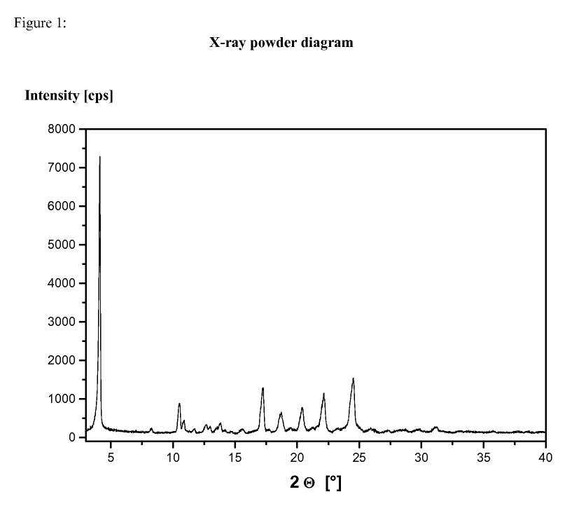

The X-ray powder diagram obtained for the salmeterol xinafoate according to the invention is shown in FIG. 1. The following Table 1 summarises the data obtained in this spectroscopic analysis:

TABLE 1
Intensities (normalised) of the X-ray reflections

| 2 Θ [°] | d [Å] | I/Iₒ [%] |
|---|---|---|
| 4.10 | 21.5 | 100 |
| 8.27 | 10.7 | 4 |
| 10.51 | 8.41 | 12 |
| 10.86 | 8.14 | 6 |
| 11.71 | 7.55 | 3 |
| 12.68 | 6.98 | 5 |
| 12.98 | 6.82 | 5 |
| 13.54 | 6.54 | 4 |
| 13.81 | 6.41 | 5 |
| 14.19 | 6.23 | 3 |
| 14.69 | 6.03 | 2 |
| 15.59 | 5.68 | 3 |
| 17.23 | 5.14 | 18 |
| 17.73 | 5.00 | 3 |
| 18.69 | 4.74 | 9 |
| 19.47 | 4.56 | 4 |
| 20.40 | 4.35 | 11 |
| 21.24 | 4.18 | 4 |
| 22.14 | 4.01 | 16 |
| 23.24 | 3.82 | 3 |
| 23.77 | 3.74 | 4 |
| 24.50 | 3.63 | 22 |
| 25.93 | 3.43 | 4 |
| 26.23 | 3.40 | 3 |
| 27.34 | 3.26 | 3 |
| 28.26 | 3.16 | 3 |
| 28.70 | 3.11 | 3 |
| 29.80 | 3.00 | 3 |
| 31.21 | 2.86 | 4 |
| 33.08 | 2.71 | 3 |
| 35.76 | 2.51 | 3 |

In the above table the value “2 Θ [°]” denotes the diffraction angle in degrees and the value “d [Å]” denotes the determined lattice plane interspacings in Å.

### II) Particle Size Determination of Micronised Tiotropium Monohydrate and Micronised Salmeterol Xinafoate:

### Measuring Instrument and Settings:

The instruments were operated in accordance with the manufacturer's operating instructions.

| | |
|---|---|
| Measuring | Laser diffraction spectrometer (HELOS), |
| instrument: | Sympatec (particle size determination by |
| | means of Fraunhofer diffraction) |
| Dispersing unit: | RODOS dry dispersing unit with suction |
| | filter, Sympatec |
| Sample amount: | 200 mg ± 150 mg |
| Product feed: | VIBRI vibrating trough, Fa. Sympatec |
| Frequency of the | increasing to 100% |
| vibrating trough: | |
| Duration of the | 15 to 25 sec. (in the case of 200 mg) |
| sample feed: | |
| Focal length: | 100 mm (measurement range: 0.9-175 μm) |
| Measurement time/ | ca. 15 sec (in the case of 200 mg) |
| waiting time: | |
| Cycle time: | 20 msec |
| Start/Stop at: | 1% at channel 28 |
| Dispersing gas: | compressed air |
| Pressure: | 3 bar |
| Reduced pressure: | maximum |
| Evaluation mode: | HRLD |

### Sample Preparation/Product Addition:

Ca. 200 mg of the test substance are weighed out on a card sheet. All relatively coarse agglomerates are broken up with a further card sheet. The powder is then scattered in finely divided form on the front half of the vibrating trough (about 1 cm from the front edge). After the start of the measurement the frequency of the vibrating trough is varied so that the addition of the sample takes place as continuously as possible. The amount of product must however also not be too large, in order for a sufficient dispersion to be achieved.

### III) Particle Size Determination of Lactose:

### Measuring Instrument and Setting

The instruments are operated in accordance with the manufacturer's operating instructions.

| | |
|---|---|
| Measuring | Laser diffraction spectrometer (HELOS), |
| instrument: | Sympatec (particle size determination by |
| | means of Fraunhofer diffraction) |
| Dispersing unit: | RODOS dry dispersing unit with suction |
| | filter, Sympatec |
| Sample amount: | 200 mg ± 100 mg |
| Product feed: | VIBRI vibrating trough, Fa. Sympatec |
| Frequency of the | increasing to 100% |
| vibrating trough: | |
| Focal length: | 200 mm (measurement range: 1.8-350 μm) |
| Measurement time/ | ca. 10 sec (in the case of 200 mg |
| waiting time: | |
| Cycle time: | 10 msec |
| Start/Stop at: | 1% at channel 28 |
| Dispersing gas: | compressed air |
| Pressure: | 3 bar |
| Reduced pressure: | maximum |
| Evaluation mode: | HRLD |

### Sample Preparation/Product Addition:

Ca. 200 mg of the test substance are weighed out on a card sheet. All relatively coarse agglomerates are broken up with a further card sheet. The powder is transferred to the edge of the vibrating trough. A distance of 1.2 to 1.4 mm between the edge of the vibrating trough and funnel is set. After the start of the measurement the amplitude setting of the vibrating trough is increased as continuously as possible to 100% towards the end of the measurement.

### IV) Determination of the Specific Surface of Micronised Tiotropium Bromide Monohydrate and Micronised Salmeterol Xinafoate (Multi-Point BET Method):

### Measuring Instrument:

TriStar 3000, Firma Micromeretics

### Sample Preparation:

Use of ½ inch sample test tubes
Weighed-out amount: ca. 1-3 g (as large as possible→do not exceed red mark)
Heating of the sample for at least 4 hours at 40° C.
Preparation station: VacPrep 061
   Method: multi-8 point p/p 0:0.05-0.12

| | |
|---|---|
| Measurement gas: | nitrogen |
| Dead volume: | to be determined by means of helium |
| Saturation pressure p0: | to be determined |
| | measurement interval: 120 min |
| | temperature: boiling point of nitrogen |
| | at ambient |
| | pressure (ca. 77 K) |
| Evacuation rate: | 50.0 mm Hg/sec |
| Evacuation time: | 0.5 hour |
| Equilibrium interval: | 15 sec |

### Implementation:

Two substance samples are measured.

The measurement values are listed individually and then averaged.

### V) Determination of the Heat of Solution of Lactose (Solution Enthalpy) E_{c}:

The solution enthalpy is measured by means of a solution calorimeter, 2225 Precision Solution Calorimeter from Fa. Thermometric.

The heat of solution is calculated on the basis of the temperature change occurring as a result of the dissolution process and the temperature change due to the system and calculated from the base line. An electrical calibration with an integrated heating resistor of accurately known output is carried out in each case before and after crushing the ampoule. A known amount of heat is thereby released to the system over a specified period of time and the rise in temperature is determined.

### Measuring Instrument and Settings

| | |
|---|---|
| Solution calorimeter: | 2225 Precision Solution Calorimeter, |
| | Fa. Thermometric |
| Reaction cell: | 100 ml |
| Thermistor resistor: | 300 kQ (at 25° C.) |
| Stirrer speed: | 500 RPM |
| Thermostat: | Thermostat of the 2277 Thermal Activity |
| | Monitor TAM, Fa. Thermometric |
| Temperature: | 25° C. ± 0.0001° C. (over 24 hours) |
| Measurement ampoules: | 1 ml crushing ampoules, Fa. |
| | Thermometric |
| Sealing: | Silicone stoppers and beeswax, Fa. |
| | Thermometric |
| Weighed-out amount: | 40 to 50 mg |
| Solvent: | Chemically pure water |
| Solvent volume: | 100 ml |
| Bath temperature: | 25° C. |
| Temperature resolution: | High |
| Start temperature: | −40 mK (±10 mK) temperature offset |
| Interface: | 2280-002 TAM accessory interface 50 |
| | Hz, Fa. Thermometric |
| Software: | SolCal V 1.1 for WINDOWS |
| Evaluation: | Automatic evaluation with menu point |
| | CALCULATION/ANALYSE EXPERIMENT. |
| | (Dynamics of the baseline; calibration |
| | after crushing the ampoule). |

### Electrical Calibration:

The electrical calibration is carried out during the measurement, once before and once after crushing the ampoule. The calibration after crushing the ampoule is used for the evaluation.

| | |
|---|---|
| Heat quantity: | 25 J |
| Heat output: | 500 mW |
| Heating duration: | 10 sec |
| Duration of the base lines: | 5 min (before and after heating) |

VI) Determination of the a_{W} Value:

### Principle of the Determination:

To determine the a_{W} value the atmospheric humidity is measured directly above a sample (water vapour partial differential pressure) after humidity equilibrium referred to 25° C. has been reached. The atmospheric humidity behaves proportionally to the a_{W} value. A meaningful a_{W} value measurement is possible only if the sample has a constant temperature during the measurement.

The determination of the atmospheric humidity, with the aid of which the a_{W} value can be calculated, must therefore be carried out in such a way that even small sample volumes, such as are present for example in the case of blister cavities, can be analysed sufficiently accurately. Specific methods for this are known to the person skilled in the art. In particular, reference may be made in the context of the present invention to the described analysis method of Xu et al. (Xu, H., Templeton, A. C., Zwierzynski, M., Mahajn R., Reed, R. A.; “Rapid, simultaneous determination of headspace oxygen and moisture in pharmaceutical packages using μGC”, Journal of Pharmaceutical and Biomedical Analysis, 38 (2005), 225-231) and to the method based on Xu et al. And described hereinafter. The measurement values given in the context of the present invention refer to the following analysis method, with the aid of which the characteristic value defined according to the invention can be determined. Alternative possible ways of achieving this object in a comparable manner can in principle also be used.

### Equipment/Material:

1.) Analysis instrument
Gas chromatography—MikroGC, Model 3000A, Manufacturer/Firma Agilent
Injection needle with side hole (supplier: Teuner Analysentechnik GmbH)
Carrier gas: helium 4.6, for example from Fa. AirLiquide
   2.) Calibration instrument
Calibration chambers for 2 sensors, manufacturer/Firma Rotronic
Certified humidity standards at 55%, 10%, 20%, 35%, 50%, 65%, 75.3%, 80%, manufacturer/Firma Rotronic
   3.) Monitoring of climatic conditions
Climatic cabinet KBF240, manufacturer/Firma Binder
Climatic measuring instrument, e.g. HygroPalm2 model with HygroClip sensor
Digital thermometer, e.g. DigiTherm model
   4.) Material for the sample preparation
Sealing of the puncture hole: Powerstrip, manufacturer/Firma Tesa
Tesafilm, manufacturer/Firma Tesa
Pin and punch

### General Information:

The measurements are carried out under constant conditions of 25° C./50% RH in a climatic cabinet.

Gas Chromatography Operating Conditions of the MikroGC, Model 3000A, Fa. Agilent:

The microGC provided with two modules is deactivated as regards module (channel)A—channel for analysis of nitrogen and oxygen—and is operated by means of module (channel) B—channel for analysis of water. It should be borne in mind that various settings, such as for example the loading time and sample inlet temperature, must however be entered for the overall system in module A. In addition reference should be made to the instrument manufacturer's instructions.

The carrier gas is provided at a pressure 5.5 bar.

TABLE 2
Tabular summary of the gas chromatography conditions:

| Module/channel/column | A | B |
|---|---|---|
| Column type | Molecular sieve | Stabilwax |
| Column dimensions | 10 m × 320 μm × | 10 m × 250 μm × |
| | 12 μm | 0.5 μm |
| Separation of | [O₂ and N₂] | [H₂O] |
| Sample inlet temperature | 90 | — |
| Injection temperature | 100 | 100 |
| Column temperature | 100 | 100 |
| Loading time | 1 | — |
| Injection time | 20 | 200 |
| Running time | 90 | 90 |
| Post-analysis time | 5 | 5 |
| Pressure equilibration time | 0 | 0 |
| Column pressure | 2.0 | 1.0 |
| Post-analysis pressure | 2.0 | 1.0 |
| Detector heating filament | Deactivated | Activated |
| Detector sensitivity | Standard | Standard |
| Detector data rate | 100 | 100 |
| Base line displacement | 0 | 0 |
| Back-flushing time | 0 | n/a |
| Chromatography duration | 1.5 | 1.5 |
| Water retention time | — | 0.90 |
| Injector type | Back-flushing | Time |
| Carrier gas | Helium 4.6 | Helium 4.6 |
| Detector type | WLD | WLD |
| Inlet type | Heated | heated |

### Calibration for the Determination of a Calibration Straight Line:

Freshly prepared calibration solutions should be used every working day. The preparation of the calibration samples (different relative humidity concentrations) is carried out according to the calibration instructions of Fa. Rotronic AG. For this purpose the humidity standards and calibration chamber must be brought to the same temperature as the micro GC. The contents of an ampoule are emptied onto the middle of the textile part in the cover of the calibration chamber and the calibration chamber is closed. After the equilibration time at least 7 injections are taken with the injection needle of the micro GC through a point sealed with Tesafilm or comparable material, according to the gas chromatography conditions described above. The first two injections are discarded, the remaining ones being used for evaluation of the plotting of the calibration straight line.

Measurement of the Sample for Determining the a_{W} Value:

The puncture point on the packaging means (for example pouch or blister cup) is provided with a “Powerstrip”, Fa. Tesa, onto which a Tesafilm is bonded. PE bottles should if necessary be punctured through the Powerstrip and Tesafilm with a punch or similar tool, the puncture site being sealed with Tesafilm immediately after this puncture operation.

The actual measurement is carried out by plunging in the injection needle of the micro GC directly after the preparation of the puncture sites according to the gas chromatography conditions (see also Table 2). Depending on the free air volume of the sample to be measured 3 to 5 injections are taken, the temperature being noted during the measurement. In this connection, for the further evaluation in each case the first injection (in the case of 3 injections) or the first two injections (in the case of more than 3 injections) should be discarded per sample being measured.

Evaluation/Calculation of the a_{W} Value:

The evaluation is made according to the calibration straight line. In this connection the a_{W} value is found as the averaged numerical value of the read-off relative humidity, divided by 100, referred to the measurement temperature (here 25° C.).

### PREPARATION OF THE POWDER FORMULATIONS ACCORDING TO THE INVENTION

### I) Apparatus

The following apparatus and equipment for example are used for the preparation of the inhalation powders:

### Mixing Container and Powder Mixer:

2 L-capacity turbulent mixer, type 2C; manufacturer Willy A. Bachofen A G, CH-4500 Basle

### Alternative Mixers:

Low-shear mixer (screw-type mixer, Ruberg mixer)
High-shear mixer (intensive mixer, Diosna-mixer)

### Hand Sieve:

0.135 mm mesh width

### Alternative Screening Devices:

Sieve granulators, for example Quadro Comil or Glatt high-speed sieve

The filling of the empty inhalation capsules with inhalation powder containing an active constituent combination can be carried out manually or by machine. The following equipment for example can be employed.

### Capsule Filling Machine:

MG2, type G100, manufacturer: MG2 S.r.1, I-40065 Pian di Macina di Pianoro (BO), Italy

### Methods for Adjusting a Specific Intrinsic Water Content (Alternatives):

Conditioning of the capsules on trays (e.g. metal drying trays from Firma Glatt, 79589 Binzen, Germany) in a climatic chamber
Conditioning of the capsules in climatic chambers, for example Weiss Climatic chamber
Conditioning of the capsules in through-flow driers, e.g. O(n)L(ine)D(ryer) BI
Conditioning of the capsules in drying containers by conditioned air
Conditioning of the capsules in drying containers by suitable preconditioned desiccants (e.g. of the zeolite, silica gel, bentonite type, etc., or of the type comprising unsaturated salts/saturated salt/salt solution systems).

Conditioning of the capsules in tightly closing bags (e.g. aluminium bags, containing suitable preconditioned desiccants (e.g. of the zeolite, silica gel, bentonite, etc., type or of the type comprising unsaturated salts, saturated salt/salt solution systems with additives, placed in wrappers that prevent escape of liquid).

### II) Experimental Examples

### Example 1

### Powder Mixture

298.63 g of excipient, 0.28 g of micronised tiotropium bromide monohydrate and 1.09 g of micronised salmeterol xinafoate are used to prepare the powder mixture. In the 300 g of inhalation powders thereby obtained the active constituent fractions are 0.09% of 1 and 0.36% of 2.

Ca. 40-45 g of excipient are added through a hand sieve with a mesh width of 0.315 mm to a suitable mixing vessel. Tiotropium bromide monohydrate 1 in portions of ca. 40-70 mg and excipient in portions of about 40-45 g are then added alternately in layers by sieving. The excipient and the auxiliary constituent 1 are added in an amount of 4 to 7 layers. The screened constituents are then mixed (mixing: 900 rpm). The final mixture is added a further two times through a hand sieve and then mixed in each case (mixing: 900 rpm).

Ca. 40-45 g of the powder mixture obtainable by the above procedure and containing the active constituent 1 are then added through a hand sieve with a mesh width of 0.315 mm to a suitable mixing vessel. Salmeterol xinafoate 2 in portions of ca. 170-250 mg and the powder mixture containing the active constituent 1 in portions of about 40-45 g are then alternately added in layers by sieving. The powder mixture containing the active constituent 1 and the active constituent 2, are added in 4 to 7 layers. The screened constituents are then mixed (mixing: 900 rpm). The final mixture is added a further two times through a hand sieve and then mixed in each case (mixing: 900 rpm).

According to or in a similar way to the procedure described in Example 1, such inhalation powders can be obtained that, after the filling of the corresponding plastics capsules, provide for example the following inhalation capsules:

### Example 2

| | | |
|---|---|---|
| Tiotropium bromide monohydrate: | 0.00937 | mg |
| Salmeterol xinafoate | 0.03632 | mg |
| Lactose monohydrate: | 5.45431 | mg |
| Polyethylene capsules: | 100.0 | mg |
| Total: | 105.5 | mg |

### Example 3

| | | |
|---|---|---|
| Tiotropium bromide monohydrate: | 0.004685 | mg |
| Salmeterol xinafoate | 0.03632 | mg |
| Lactose monohydrate: | 5.958995 | mg |
| Polyethylene capsules: | 100 | mg |
| Total: | 106 | mg |

### Example 4

| | | |
|---|---|---|
| Tiotropium bromide monohydrate: | 0.00625 | mg |
| Salmeterol xinafoate | 0.03632 | mg |
| Lactose monohydrate: | 9.95743 | mg |
| Polyethylene capsules: | 100 | mg |
| Total: | 110 | mg |

### Example 5

| | | |
|---|---|---|
| Tiotropium bromide monohydrate: | 0.0125 | mg |
| Salmeterol xinafoate | 0.07264 | mg |
| Lactose monohydrate: | 9.91486 | mg |
| Polyethylene capsules: | 100 | mg |
| Total: | 110 | mg |

### Example 6

### Powder Mixture (Composition)

298.63 g of excipient (95% lactose monohydrate “Pharmatose 200M”, manufacturer DMV+5% micronised lactose monohydrate), 0.28 g micronised tiotropium bromide monohydrate and 1.09 g micronised salmeterol xinafoate are used for the preparation of the powder mixture. In the 300 g of inhalation powder thereby obtained the active constituent fraction is 0.09% of 1 and 0.36% of 2.

### Excipient Mixture:

283.7 g of Pharmatose 200M are used as coarser excipient component. 14.93 of micronised lactose monohydrate are used as finer excipient component. In the 298.63 g of excipient mixture obtained in this way the proportion of finer excipient component is 5%.

Ca. 7.5 g to 11.3 g of Pharmatose 200M are added through a suitable sieve granulator containing a screen with a mesh width of 0.5 mm to a suitable mixing vessel. Micronised lactose monohydrate in portions of ca. 2 to 5 g and Pharmatose 200 M in portions of 2 to 5 g are then added alternately in layers through the screen. Pharmatose 200M and micronised lactose monohydrate are added in 31 and 30 layers respectively (tolerance: ±6 layers).

The screened constituents are then mixed (mixing: 900 rpm).

### Final Mixture:

Ca. 40-45 g of excipient mixture are added through a hand sieve having a machine width of 0.315 mm to a suitable mixing vessel. Salmeterol xinafoate 2 in portions of ca. 90-110 mg and excipient in portions of about 40-45 g as well as tiotropium 1 in portions of ca. 22-28 mg are then added alternately in layers through the screen. The addition of the excipient mixture and of the active constituents 2 and 1 takes place in 8 to 11 layers. The screened constituents are then mixed (mixing: 900 rpm). The final mixture is also added twice through a hand sieve and is in each case mixed (mixing: 900 rpm).

According to or in a similar way to the procedure described in Example 6, inhalation powders can be obtained that, after the filling of the corresponding plastics capsules, provide for example the following inhalation capsules:

### Example 7

| | | |
|---|---|---|
| Salmeterol xinafoate | 0.03632 | mg |
| Tiotropium bromide monohydrate: | 0.0125 | mg |
| Lactose monohydrate mixture: | 5.46368 | mg |
| Polyethylene capsules: | 100 | mg |
| Total: | 105.5 | mg |

### Example 8

| | | |
|---|---|---|
| Salmeterol xinafoate | 0.07264 | mg |
| Tiotropium bromide monohydrate: | 0.00625 | mg |
| Lactose monohydrate mixture: | 9.92736 | mg |
| Polyethylene capsules: | 100 | mg |
| Total: | 110 | mg |

### Example 9

| | | |
|---|---|---|
| Salmeterol xinafoate | 0.07264 | mg |
| Tiotropium bromide monohydrate: | 0.004685 | mg |
| Lactose monohydrate mixture: | 19.92736 | mg |
| Polyethylene capsules: | 100 | mg |
| Total: | 120.0 | mg |

### Example 10

### Powder Mixture

298.63 g of excipient (lactose monohydrate “Pharmatose 200M”, manufacturer DMV), 0.28 g of micronised tiotropium bromide monohydrate and 1.09 g of micronised salmeterol xinafoate are used for the preparation of the powder mixture. In the 300 g of inhalation powder thereby obtained the active constituent proportions are 0.09% of 1 and 0.36% of 2.

Ca. 40-45 g of excipient are added through a hand sieve with a mesh width of 0.315 mm to a suitable mixing vessel. Tiotropium bromide monohydrate 1 in portions of ca. 40-70 mg and excipient in portions of about 40-45 g are then added alternately in the form of layers. The addition of the excipient and of the active constituent 1 takes place in 4 to 7 layers.

The screened constituents are then mixed (mixing: 900 rpm). The final mixture is also added twice through a hand sieve and is then mixed in each case (mixing: 900 rpm).

Ca. 40-45 g of the powder mixture obtained by the procedure described above and containing the active constituent 1 are then added through a hand sieve with a mesh width of 0.315 mm to a suitable mixing vessel. Following this salmeterol xinafoate 2 in portions of ca. 170-250 mg and the powder mixture containing the active constituent 1 in portions of about 40-45 g are added alternately in layers through the sieve. The addition of the powder mixture containing the active constituent 1 and of the active constituent 2 takes place in 4 to 7 layers.

The screened constituents are then mixed (mixing: 900 rpm). The final mixture is also added twice through a hand sieve and then mixed in each case (mixing: 900 rpm).

According to or in a similar way to the procedure described in Example 1, inhalation powders can be obtained that, after the filling of the corresponding plastics capsules, provide for example the following inhalation capsules:

### Example 11

| | | |
|---|---|---|
| Tiotropium bromide monohydrate: | 0.00937 | mg |
| Salmeterol xinafoate | 0.03632 | mg |
| Lactose monohydrate: | 9.95431 | mg |
| Polyethylene capsules: | 100 | mg |
| Total: | 110.0 | mg |

### Example 12

| | | |
|---|---|---|
| Tiotropium bromide monohydrate: | 0.00625 | mg |
| Salmeterol xinafoate | 0.03632 | mg |
| Lactose monohydrate: | 9.95743 | mg |
| Polyethylene capsules: | 100 | mg |
| Total: | 110 | mg |

### Example 13

### Powder Mixture

5725.86 g of excipient, 56.22 g of micronised tiotropium bromide monohydrate and 217.92 g of micronised salmeterol xinafoate are used to prepare the powder mixture. In the 6 kg of inhalation powder thereby obtained the proportions of active constituents are 0.0937% of 1 and 0.363% of 2.

Ca. 50% of the excipient lactose monohydrate of Respitose ML003 (200M) quality, obtained from DMV, are added by means of a sieve granulator from Fa. Glatt, GS180 with a mesh width of 0.45 mm (rotor speed 900 rpm) to a suitable mixing vessel. Tiotropium bromide monohydrate 1 in one portion followed by ca. 500 g of the excipient and then the total amount of 2 followed by ca. 500 g of the excipient are then added alternately in layers through the sieve. This is followed by the addition of the residual amount of the excipient through the sieve granulator to the mixing vessel. The screened constituents are then mixed for 8 minutes (stirrer speed 90 rpm, chopper speed 1500 rpm).

According to or in a similar way to the procedure described in Example 1, inhalation powders can be obtained that, after the filling of the corresponding plastics capsules, provide for example the following inhalation capsules:

### Example 14

| | | |
|---|---|---|
| Tiotropium bromide monohydrate: | 0.00937 | mg |
| Salmeterol xinafoate | 0.03632 | mg |
| Lactose monohydrate: | 9.95431 | mg |
| Polyethylene capsules: | 100 | mg |
| Total: | 110.0 | mg |

### Example 15

| | | |
|---|---|---|
| Tiotropium bromide monohydrate: | 0.009996 | mg |
| Salmeterol xinafoate | 0.043587 | mg |
| Lactose monohydrate: | 9.946417 | mg |
| Polyethylene capsules: | 100 | mg |
| Total: | 110.0 | mg |

### Example 16

| | | |
|---|---|---|
| Tiotropium bromide monohydrate: | 0.0087465 | mg |
| Salmeterol xinafoate | 0.0334167 | mg |
| Lactose monohydrate: | 9.95784 | mg |
| Polyethylene capsules: | 100 | mg |
| Total: | 110 | mg |

### Example 17

| | | |
|---|---|---|
| Tiotropium bromide monohydrate: | 0.004685 | mg |
| Salmeterol xinafoate | 0.03632 | mg |
| Lactose monohydrate: | 9.958995 | mg |
| Polyethylene capsules: | 100 | mg |
| Total: | 110.0 | mg |

### Example 18

| | | |
|---|---|---|
| Tiotropium bromide monohydrate: | 0.006247 | mg |
| Salmeterol xinafoate | 0.03632 | mg |
| Lactose monohydrate: | 9.957433 | mg |
| Polyethylene capsules: | 100 | mg |
| Total: | 110.0 | mg |

### Example 19

| | | |
|---|---|---|
| Tiotropium bromide monohydrate: | 0.012493 | mg |
| Salmeterol xinafoate | 0.03632 | mg |
| Lactose monohydrate: | 9.951187 | mg |
| Polyethylene capsules: | 100 | mg |
| Total: | 110.0 | mg |

### PREPARATION OF THE FILLED AND PACKAGED MEDICAMENTS ACCORDING TO THE INVENTION CONTAINING INHALATION POWDERS ACCORDING TO THE INVENTION

### Preparation of Preconditioned Desiccant Bags for the Examples A to D

The conditioning is carried out in a suitable climatic cabinet.

The desiccant silica gel in 25 g and 2 g Tyvek bags is placed, without overlapping, on drying trays (e.g. metal drying trays from Firma Glatt, 79589 Binzen, Germany). When the metal drying trays are brought on a tray trolley into the climatic chamber a distance of at least 25 cm should be left between the metal sheets.

| Process data | Desired | Tolerances |
|---|---|---|
| Chamber climate: | 25° C. | 23° C.-27° C. |
| | 25% RH | 20%-30% RH* |
| Conditioning time | 96 hours | 90-126 hours |

### Example A

Inhalation powders corresponding to the Examples 1, 5, 10 or 13 are distributed in a thickness of at most 1 cm on metal trays. The inhalation powder should be conditioned for at least 24 hours at 25° C.+/−2° C./25%+/−3% relative humidity in a climatic chamber or climatic cabinet. The inhalation powder is packed in hermetically sealable steel containers under these climatic conditions.

Inhalation powder preconditioned according to the invention is packed directly in a blister cup and sealed, the forming of the blister cup, the filling and the sealing process being carried out in a climatically controlled work room (22°-28° C./20-30% relative humidity).

Such blisters (corresponding to the primary packaging means) are in addition provided with pouches, wherein in this packaging step a 2 g desiccant bag containing silica gel preconditioned at 25% relative humidity (referred to 25° C.) in a Tyvek bag (quality: Sorb-It®, 2 g, Fa. Süd Chemie AG, D-85368 Moosburg) is placed in the pouch (corresponding to the secondary packaging means).

### Example B

Similarly to the procedure described in Example A, such medicament blisters that contain preconditioned inhalation powders can also be placed directly in inhalation devices. Following this the inhalation device is provided with pouches, wherein a 2 g desiccant bag containing silica gel preconditioned at 25% relative humidity (referred to 25° C.) in a Tyvek bag (quality: Sorb-It®, 2 g, Fa. Süd Chemie AG, D-85368 Moosburg) is placed in the pouch (corresponding to the secondary packaging means).

### Example C

Powder capsules for inhalation that are produced according to Examples 2 to 5, 6 to 9, 11 or 12, or 14 to 19 are packed in a hermetically sealable sealed container so that the latter is filled to 30% to at most 75% of the filling volume. One desiccant bag of quality Sorb-It®, 25 g, Fa. Süd Chemie AG, D-85368 Moosburg, containing silica gel preconditioned at 25% relative humidity (referred to 25° C.) in a Tyvek bag, per 100,000 capsules is transferred to an insert in the container so that a free gas exchange can take place within the container. The container is sealed and stored at 25° C. for at least 5 days to reach an equilibrium humidity.

After completion of the conditioning of the capsules the latter are packed in a climatically controlled work room (20°-28° C./20-30% relative humidity) in PE flasks (corresponding to the primary packaging means), which contain a preconditioned (20-25% relative humidity at 25° C.) desiccant cartridge in the cover.

### Example D

Similarly to the procedure described in Example C, medicaments containing inhalation capsules that have been conditioned in accordance with the container conditioning according to Example C can also be packaged in blisters according to the invention (corresponding to the primary packaging means). The conditioned capsules are in this connection blister-packed in a climatically controlled work room (22°-28° C./20-30% relative humidity) or in an encapsulated blister pack machine that maintains the above climatic conditions (22°-28° C./20-30% relative humidity). In this connection blister films are used that have preferably been kept for at least 12 months at 20°-28° C./20-30% relative humidity.

Such blisters are in addition provided with pouches, wherein in this packaging step a 2 g desiccant bag containing silica gel preconditioned at 25% relative humidity (referred to 25° C.) in a Tyvek bag (quality: Sorb-It®, 2 g, Fa. Süd Chemie AG, D-85368 Moosburg) is placed in the pouch (corresponding to the secondary packaging means).

### III) Examples of the Demonstration of the Suitability of Inhalation Powders According to the Invention as Inhalative Medicament

The particular suitability of the inhalation powders according to the invention can be comprehensively demonstrated for example by testing the powders for their inhalative performance and the decomposition rate of the active constituent as a function of time.

The inhalative performance can in this connection be determined by analysing the fine particle fraction (FPF) of the medicament. The FPF is understood to mean the inhalable dose (particles <5 μm) that can be determined on the basis of Pharm. Eur. 2.9.18 (European Pharmacopoeia, 6th edition 2008, Apparatus D—Andersen Cascade Impactor) and USP30-NF25 <601>, and is related to the nominal active constituent dose of the tested amount (dose). The result is consequently given in %.

The testing (according to the following Examples I and II) was carried out according to the invention using inhalation powders that had been produced according to Example 14 and discharged with an inhaler corresponding to FIG. 2.

### Example I

FPF results (for tiotropium) of four batches are shown in FIG. 3. From each of the four batches inhalation capsules, such as are obtainable according to Example 14, were on the one hand packaged directly after the production of the capsules, and on the other hand were brought into equilibrium with the ambient humidity, so that the a_{W} value, as a characteristic quantity of the inhalation powder, was less than 0.05 (referred to 25° C.). The sample that had been analysed after preparation without any further subsequent treatment was in equilibrium with an a_{W} value between 0.45 and at most 0.55 (referred to 25° C.).

In this connection it is found that inhalation powder containing a salmeterol salt and a tiotropium salt, and which are in equilibrium with an a_{W} value of less than 0.05, have a lower FPF than when they are brought into equilibrium with an a_{W} value between 0.45 and 0.55 (referred to 25° C.). The FPF (tiotropium) for the product that is characterised by an a_{W} value of 0.45 to 0.55 is more than 30%. The FPF for the product that is characterised by an a_{W} value of less than 0.05 is less than 30%.

A fall/difference of the FPF of around 20% (referred to the higher FPF value) is observed.

### Example II

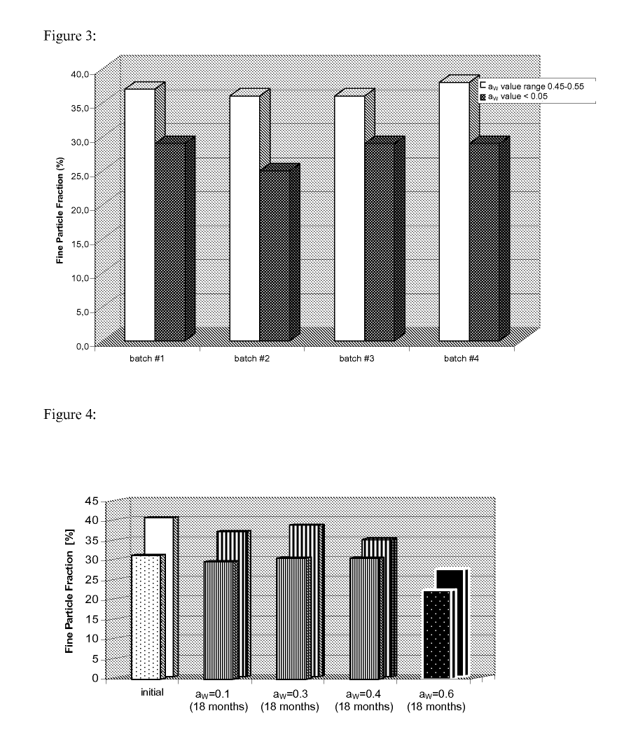

Results of the FPF of salmeterol and of tiotropium from a batch such as is obtainable according to Example 14 are shown in FIG. 4. The front row of bars corresponds to the FPF of tiotropium, while the rear row of bars corresponds to that of salmeterol. The product was initially brought to an equilibrium humidity, so that the a_{W} value was between 0.28 (and?) (referred to 25° C.). The initial FPF for tiotropium and salmeterol from this batch correspond to an a_{W} value of 0.28. To check the stability, the product was exposed to conditions so that the inhalation powder in the capsules was in equilibrium with an ambient humidity corresponding to an a_{W} value of 0.1, 0.3, 0.4 and 0.6 (referred to 25° C.).

The initial FPF of salmeterol (a_{W} value=0.28) was ca. 40%. The FPF of salmeterol (a_{W} value=0.1, 0.3 and 0.4) was after 18 months' storage more than 30%. The initial FPF of tiotropium (a_{W} value=0.28) was ca. 30%. The FPF of tiotropium (a_{W} value=0.1, 0.3 and 0.4) was after 18 months' storage more than 25%. The FPF of salmeterol (a_{W} value=0.6) was after 18 months' storage less than 30%. The FPF of tiotropium (a_{W} value=0.6) was after 18 months' storage less than 25%.

| | FPF [%] | | | | |
|---|---|---|---|---|---|
| | | aW = 0.1 | aW = 0.3 | aW = 0.4 | aW = 0.6 |
| | initial | (18 months) | (18 months) | (18 months) | (18 months) |
| FPF Value [%] - Sal | 40 | 37 | 38 | 35 | 27 |
| FPF value[%] - Tio | 31 | 30 | 31 | 31 | 23 |

Whereas the FPF for tiotropium and also salmeterol were after 18 months comparable to the initial value, a decrease in the FPF of more than 25% (tiotropium) and more than 30% (salmeterol) was observed (in each case referred to the initial value).

### Example III

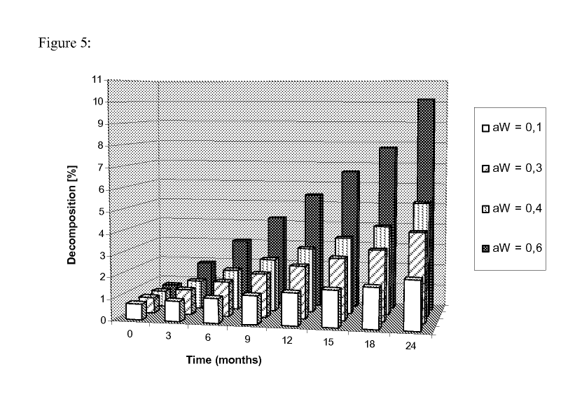

Results of the decomposition behaviour of salmeterol of a batch such as is obtainable according to Example 14 are shown in FIG. 5. To check the stability the product was exposed to conditions so that the inhalation powder in the capsules was in equilibrium with an ambient humidity corresponding to an a_{W} value of 0.1, 0.3, 0.4 and 0.6 (referred to 25° C.). It is found that an acceptable decomposition behaviour over a period of at least 18 months can be ensured only if the product is maintained in equilibrium with the humidity (measured directly above the product), which corresponds to an a_{W} value of 0.1, 0.3 or 0.4.

1. Inhalation powder containing the excipient lactose, an acid addition salt of salmeterol 2 and a tiotropium salt 1, characterised in that it has an a_{W} value between 0.05 and 0.5.

2. Inhalation powder according to claim 1, characterised in that it has an a_{W} value between 0.1 and 0.45.

3. Inhalation powder according to claim 1, characterised in that it has an a_{W} value between 0.1 and 0.4.

4. Inhalation powder according to claim 1, wherein the acid addition salt of salmeterol is salmeterol xinafoate, characterised by a melting point of 124° C., as well as tiotropium in combination with a corresponding counterion selected from the group chloride, bromide, iodide, methanesulfonate or para-toluenesulfonate, preferably tiotropium bromide monohydrate.

5. Inhalation powder according to claim 1, characterised in that the tiotropium salt 1 and salmeterol salt 2 are contained jointly in doses of 5 to 5000 μg.

6. Inhalation powder according to claim 1, characterised in that the tiotropium 1′ is contained in a dose of 3.8 μg to 15 μg and salmeterol xinafoate 2′ is contained in a dose of 12.5 to 50 μg.

7. Inhalation powder according to claim 1, characterised in that the mean particle size ×50 is between 15 μm and 65 μm with a 10% fines fraction of 1 to 8 μm.

8. Predosed inhalation powder according to claim 1, characterised in that the dose contains 1 to 20 mg, preferably about 3 to 15 mg of inhalation powder.

9. Predosed inhalation powder according to claim 1, characterised in that the dose contains 8 to 12 mg of inhalation powder.

10. Predosed inhalation powder according to claim 8 characterised in that the FPF of the salmeterol salt is at least 30%.

11. Predosed inhalation powder according to claim 8 characterised in that the FPF of the tiotropium salt is at least 25%.

12. Predosed inhalation powder according to claim 8 characterised in that the overall decomposition of salmeterol after 18 months' storage in the packaging means (25° C. and 60% relative humidity) is at most 5%, the decrease in the FPF of the salmeterol salt referred to the initial FPF value after 18 months' storage in the packaging means is less than 30%, and the decrease in the FPF of the tiotropium salt referred to the initial FPF after 18 months' storage in the packaging means (25° C. and 60% relative humidity) is less than 25%.

13. Predosed inhalation powder according to claim 8 characterised in that the overall decomposition of salmeterol after 18 months' storage in the packaging means (25° C. and 60% relative humidity) is at most 5%, the decrease in the FPF of the salmeterol salt referred to the initial FPF after 18 months' storage in the packaging means (25° C. and 60% relative humidity) is less than 20%, and the decrease in the FPF of the tiotropium salt referred to the initial FPF after 18 months' storage in the packaging means (25° C. and 60% relative humidity) is less than 20%.

14. Predosed inhalation powder according to claim 8 characterised in that the interface between the inhalation powder and the dose container is selected from a material consisting of synthetic plastics.

15. Predosed inhalation powder according to claim 8 characterised in that the dose container is made of a non-hygroscopic material that can absorb or release less than 0.5% (w/w) water.

16. Predosed inhalation powder according to claim 8 characterised in that the dose container is made of a material that has an electrical conductivity sigma that is less than 10⁻⁵ S cm⁻¹.

17. Inhalation powder according to claim 8 predosed in a capsule, wherein the capsule consists of a synthetic plastic.

18. Packaged medicament containing an inhalation powder according to claim 4 characterised in that the packaging means contains, apart from the inhalation powder, a pre-moisturised desiccant so that an a_{W} value between 0.05 and 0.5 prevails in the packaging means.

19. Packaged medicament containing an inhalation powder according to claim 4 characterised in that the packaging means contains, apart from the inhalation powder, a pre-moisturised desiccant so that an a_{W} value between 0.1 and 0.45 prevails in the packaging means.

20. Packaged medicament containing an inhalation powder according to claim 4 characterised in that the packaging means contains, apart from the inhalation powder, a pre-moisturised desiccant so that an a_{W} value between 0.1 and 0.4 prevails in the packaging means.

21. Packaged medicament containing an inhalation powder according to claim 4 characterised in that the packaged inhalation powders are double packaged, and an a_{W} value between 0.05 and 0.5 prevails within the primary packaging means and also between the primary packaging means and secondary packaging means.

22. Packaged medicament containing an inhalation powder according to claim 4 characterised in that the packaged inhalation powders are double packaged, and an a_{W} value between 0.1 and 0.45 prevails within the primary packaging means and also between the primary packaging means and secondary packaging means.

23. Packaged medicament containing an inhalation powder according to claim 4 characterised in that the packaged inhalation powders are double packaged, and an a_{W} value between 0.1 and 0.4 prevails within the primary packaging means and also between the primary packaging means and secondary packaging means.

24. Packaged medicament containing an inhalation powder according to claim 4 characterised in that the packaged inhalation powders are double packaged, wherein the secondary packaging means contains apart from the primary-packaged medicament also a pre-moisturised desiccant, so that an a_{W} value between 0.05 and 0.5 prevails therein.

25. Packaged medicament containing an inhalation powder according to claim 4 characterised in that the packaged inhalation powders are double packaged, wherein the secondary packaging means contains apart from the primary-packaged medicament also a pre-moisturised desiccant, so that an a_{W} value between 0.15 and 0.45 prevails therein.

26. Packaged medicament containing an inhalation powder according to claim 4 characterised in that the packaged inhalation powders are double packaged, wherein the secondary packaging means contains apart from the primary-packaged medicament also a pre-moisturised desiccant, so that an a_{W} value between 0.1 and 0.4 prevails therein.

27. Packaged medicament according to claim 22 wherein the secondary packaging means forms a pouch system.

28. Packaged medicament according to claim 18 or 24 wherein the pre-moisturised desiccant is selected from the group consisting of silica gel, molecular sieves or bentonite.

29. Packaged medicament according to claim 28, wherein the pre-moisturised desiccant before use is brought into equilibrium with its water load, in terms of 20-30% relative humidity (referred to 23° to 27° C.).

30. Packaged medicament according to claim 28 wherein the pre-moisturised desiccant is a silica gel contained in a vapour-permeable bag.

31. Packaged medicament according to claim 18 wherein the predosed inhalation powders are packaged in blisters as primary packaging means.

32. Process for the preparation of inhalation powders containing lactose, a salmeterol salt and a tiotropium salt, characterised in that the inhalation powder is exposed to a humidity so that an a_{W} value between 0.05 and 0.5 is in equilibrium above the inhalation powder.

33. Process according to claim 32 for the preparation of inhalation powders containing lactose, a salmeterol salt and a tiotropium salt, characterised in that the inhalation powder is exposed to a humidity so that an a_{W} value between 0.1 and 0.45 is in equilibrium above the inhalation powder.

34. Process according to claim 32 for the preparation of inhalation powders containing lactose, a salmeterol salt and a tiotropium salt, characterised in that the inhalation powder is exposed to a humidity so that an a_{W} value between 0.1 and 0.4 is in equilibrium above the inhalation powder.

35. Process according to claim 32 characterised in that the inhalation powders
a. are conditioned for at least 4 hours at a relative humidity of 18 to 27% and at 16° C. to 28° C.
b. the predosed inhalation powders are then packaged in an ambient humidity of 20 to 30% and at a temperature of 23 to 28° C.

36. Process according to claim 35, characterised in that for the packaging of the predosed inhalation powders a packaging material is used that is characterised by a permeation rate for water of less than 5 g/m²/d.

37. Process according to claim 35 characterised in that predosed inhalation powders are packed in capsules for inhalation and the packaging means exists in the form of a blister.

38. Process for the preparation of packaged medicaments for inhalation according to claim 35 comprising the following steps
a. preparation of predosed inhalation powders according to claims 32
b. following which the primary-packaged inhalation powders are secondarily packaged in an ambient humidity of 20 to 30% and at a temperature of 23 to 28° C.

39. Process according to claim 38, characterised in that for the secondary packaging of the primary-packaged inhalation powders, a packaging material is used that is characterised by a water vapour permeability of less than 5 g/m²d.

40. Process according to 38 characterised in that a pre-moisturised desiccant is placed in the secondary packaging means together with the primary-packaged, predosed inhalation powder.

41. Process according to claim 40, characterised in that the pre-moisturised desiccant is selected from silica gel, molecular sieves or bentonite and is exposed for at least 24 hours before use to a relative humidity of 20-30% (referred to 23° to 27° C.).

42. Medicaments that are produced by a method according to claim 32.

43. Inhalation kit containing an inhalation device that can be used for administering inhalation powders from powder-containing capsules, and a medicament according to claim 1 or 42.

44. Inhalation kit according to claim 43, characterised in that the inhaler is characterised by a housing 1 containing two windows 2, a cover 3 in which are located air inlet openings and which is provided with a sieve 5 secured via a sieve housing 4, an inhalation chamber 6 connected to the cover 3, on which chamber is provided a plunger 9 movable against a spring 8 and provided with two ground needles 7, a mouthpiece 12 pivotable on a shaft 10 and connected to the housing 1, the cover 3 and to a cap 11, as well as air passage holes 13 for adjusting the flow resistance.
